(19) United States
(12) Patent Application Publication
(10) Pub. No.: US 2025/0389711 A1
(43) Pub. Date: Dec. 25, 2025

(54) Advanced Method for Accurate Optical Quantification of Antioxidant Carotenoids in Biological Tissues with Comprehensive Correction Techniques

(71) Applicant: Mohsen Sharifzadeh, Cottonwood Heights, UT (US)

(72) Inventor: Mohsen Sharifzadeh, Cottonwood Heights, UT (US)

(21) Appl. No.: 18/749,255

(22) Filed: Jun. 20, 2024

(51) Int. Cl. G01N 33/483; G01N 21/31; G01N 33/49

(52) U.S. Cl. CPC G01N 33/4833; G01N 21/31; G01N 33/49

(57) ABSTRACT

This patent describes an advanced method for the accurate optical quantification of antioxidant carotenoids in biological tissues. The method addresses interference from melanin and hemoglobin through comprehensive correction techniques. It involves creating a tangent line for melanin correction using absorbance values at 650 nm and 700 nm, measuring the corrected optical density (OD) of carotenoids at 488 nm, measuring the corrected OD of blood at 577 nm, converting the Blood OD from 577 nm to 488 nm using oxyhemoglobin and deoxyhemoglobin extinction coefficients, and subtracting the Blood OD from the Carotenoid OD to obtain the final Carotenoid OD. This innovative approach provides reliable, non-invasive, and precise measurements of carotenoid levels in tissues, facilitating improved health assessments and personalized nutrition strategies.

To address these challenges, we propose an advanced method that combines comprehensive correction techniques, including melanin and hemoglobin correction, BMI or weight adjustment, and standardized sampling on the palm. Additionally, we integrate temperature compensation, calibration with standard references, multi-spectral analysis, and advanced noise reduction techniques to enhance measurement accuracy. Machine learning models are also employed to refine the measurements and account for individual variability.

This innovative approach promises to overcome the limitations of traditional methods, providing a reliable and precise tool for the optical quantification of antioxidant carotenoids in biological tissues. By leveraging these advanced techniques, we aim to improve the accuracy of carotenoid measurements, facilitating better health assessments and personalized nutrition strategies.

### Detailed Methodology

### Melanin Correction

### Tangent Baseline Creation:

Absorbance values at 650 nm and 700 nm are used to create a tangent baseline.

This baseline corrects for melanin interference in the absorbance spectrum.

### Correction Application:

The baseline is subtracted from the smoothed absorbance spectrum to obtain the melanin-corrected spectrum.

### Carotenoid Measurement

### Measurement of Carotenoid OD:

The melanin-corrected absorbance at 488 nm is measured to obtain the Carotenoid OD.

### Blood Correction

### Measurement of Blood OD:

The melanin-corrected absorbance at 577 nm is measured to obtain the Blood OD.

### Conversion to Target Wavelength:

The Blood OD at 577 nm is converted to the equivalent value at 488 nm using the following equation:

$$Blood {OD}_{488} = Blood {OD}_{577} \times ( 0.95 \times \frac{ext - coeff - {oxy}_{488}}{ext - coeff - {oxy}_{577}} + 0.05 \times \frac{ext - coeff - {deoxy}_{488}}{ext - coeff - {deoxy}_{577}} )$$

### Final Carotenoid OD Calculation:

The Blood OD at 488 nm is subtracted from the Carotenoid OD at 488 nm to obtain the final Carotenoid OD.

### FIGURES AND DESCRIPTIONS

FIG. 1: Smoothed Optical Absorbance Spectrum

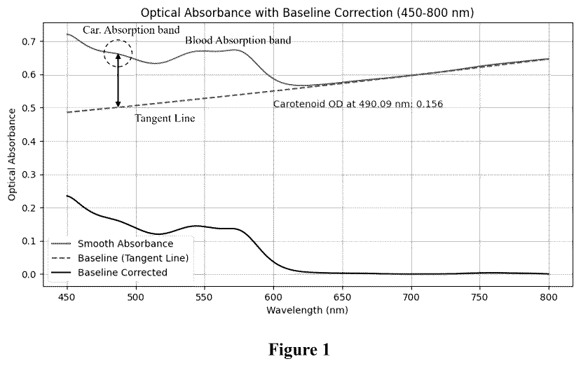

This figure illustrates the smoothed optical absorbance spectrum of a tissue sample within the visible light spectrum, highlighting the peaks corresponding to carotenoids.

FIG. 2: Corrected Optical Absorbance Spectrum with Melanin and Blood Corrections

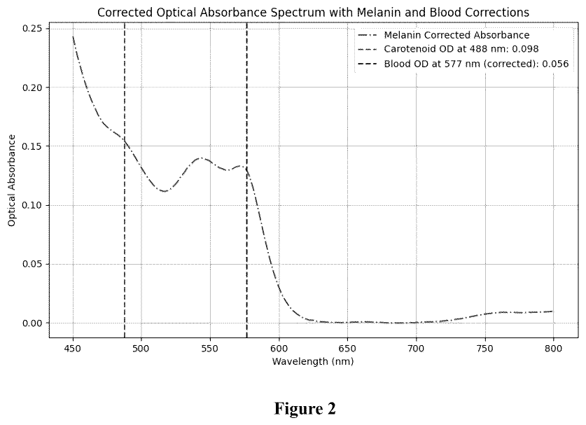

The measured absorbance spectrum is shown after correction for melanin and blood interference. The melanin correction is applied using a tangent line between 650 nm and 700 nm. The Carotenoid OD is measured at 488 nm (red dashed line), and the Blood OD is measured at 577 nm (blue dashed line). The Blood OD at 577 nm is converted to the equivalent value at 488 nm using the oxyhemoglobin extinction coefficients, assuming 95% oxyhemoglobin and 5% deoxyhemoglobin. The final Carotenoid OD at 488 nm is calculated by subtracting the Blood OD at 488 nm.

1: Preamble: A method for measuring carotenoid levels in tissue.
Transitional Phrase: Comprising:
Claim Body:
Creating a baseline for melanin correction using absorbance values within a specified range;
Measuring the corrected optical density (OD) of carotenoids at a target wavelength within a defined spectral range;
Converting the Blood OD from a known blood absorption peak to the target wavelength using extinction coefficients;
Subtracting the Blood OD from the Carotenoid OD to obtain the final Carotenoid OD.

2: Preamble: A method for correcting melanin interference in optical absorbance measurements.
Transitional Phrase: Comprising:
Claim Body:
Using absorbance values at two points within a specified spectral range to create a tangent baseline;
Applying the baseline to correct absorbance measurements for melanin interference.

3: Preamble: A method for correcting blood interference in optical absorbance measurements.
Transitional Phrase: Comprising:
Claim Body:
Measuring blood OD at a known absorption peak;
Converting the blood OD to a target wavelength using appropriate extinction coefficients;
Subtracting the converted blood OD from the absorbance measurement at the target wavelength.

4: Preamble: The method of claim 1.
Claim Body: Further comprising adjusting the optical density readings based on body size, such as using a correction factor derived from BMI, weight, or other relevant biometric data.

5: Preamble: The method of claim 1.
Claim Body: Further comprising standardizing the sampling area in optical measurements, such as by using a standardized device or protocol to maintain uniformity across all subjects.

6: Preamble: The method of claim 1.
Claim Body: Further comprising integrating machine learning models trained on datasets that include variables such as melanin levels, hemoglobin concentration, biometric data, and actual carotenoid levels.

7: Preamble: The method of claim 1.
Claim Body: Further comprising compensating for environmental variations in optical measurements by measuring the environmental factors during the optical measurement and applying appropriate correction factors.

8: Preamble: The method of claim 1.
Claim Body: Further comprising regularly calibrating the optical device with standard references using standard reference materials with known optical properties.

9: Preamble: The method of claim 1.
Claim Body: Further comprising utilizing multi-spectral analysis in optical measurements by measuring absorbance across a broad range of wavelengths and analyzing the spectral data to differentiate carotenoids from other compounds.

10: Preamble: The method of claim 1.
Claim Body: Further comprising applying advanced noise reduction techniques in optical measurements to enhance the clarity and accuracy of the OD readings.

11. Preamble: The method of claim 1.
Claim Body: Further comprising adjusting measurements based on tissue hydration status by assessing the hydration status of the tissue during measurement and applying a correction factor to adjust for hydration-related variations in OD readings.
